(19) United States
(12) Patent Application Publication
(10) Pub. No.: US 2009/0318935 A1
(43) Pub. Date: Dec. 24, 2009

(54) Percutaneous medical devices and methods

(76) Inventors: Satish Sundar, Milpitas, CA (US); Thyagarajan Subramanian, Hummelstown, PA (US)

Correspondence Address: LAW OFFICE OF MARK WISNOSKY, 3200 Fourth Avenue, Suite 201, SAN DIEGO, CA, 92103, US

(21) Appl. No.: 12/092,428

(22) PCT Filed: Nov. 10, 2006
(86) PCT No.: PCT/US06/60809

§ 371 Date: May 2, 2008

Related U.S. Application Data
(60) Provisional application No. 60/735,427, filed on Nov. 10, 2005.
(60) Provisional application No. 60/755,055, filed on Dec. 30, 2005.
(60) Provisional application No. 60/792,996, filed on Apr. 18, 2006.

(51) Int. Cl. A61B 19/00; A61B 1/32

(52) U.S. Cl. 606/130; 600/201

(57) ABSTRACT

Apparatus and methods are described providing improved surgical and similarly invasive procedures. The components may be used individually or collectively as a system. They consist of new apparatus for insertion of transducers and transducer arrays into the body cavity, apparatus and procedures for controlling pressure differential between the body cavity and the atmosphere, imaging techniques to combine the now available real-time images with pre-procedure imaging, and robotic placement techniques that when combined with all of the above provide improved surgical and medical procedures.

### CROSS-REFERENCE TO RELATED APPLICATIONS

This application claims the benefit of U.S. Provisional Patent Application 60/735,427, filed Nov. 10, 2005, entitled, “Percutaneous transcranial real-time multimodality imaging guided stereotactic interventional devices and methods” and U.S. Provisional Patent Application 60/755,055, filed Dec. 30, 2005, entitled “Additional Methods for Real-Time Percutaneous Stereotactic Multimodality Assessment and Intervention” and U.S. Provisional Patent Application 60/792,996, filed Apr. 18, 2006, entitled “More Methods for Real-Time Percutaneous Transcranial Stereotactic Multimodality Assessment and Intervention”, all currently pending, by the same inventors, and, incorporated by reference.

### TECHNICAL FIELD

Embodiments of the invention relate to apparatus and procedures suitable for use in percutaneous medical and surgical procedures.

### BACKGROUND OF THE INVENTION

Current medical and surgical procedures utilize pre-procedural imaging to map the body interior and subsequent mathematical calculation of geometric targets within the body interior during and immediately pre-procedure. The effects of the procedure are evaluated by post-procedural imaging to verify accuracy and also by the effects of the procedure itself. Current procedures are limited by their inability to provide real time imaging. Thus, these procedures are fraught with significant imaging limitation posed by bone matter, physical constraints of magnetic resonance imaging and computer tomography scanners that are used for routine imaging, inability to use metal instruments in high magnetic fields and the lack of true real time images to act as guides during the procedures. Further, introduction of air into the body at the time of surgery, resulting in a significant shift of the internal organs, and, technical errors in the placement of stereotactic and other surgical or medical frames and guides result in inaccuracies in procedures. The aforementioned limitations compromise the accuracy of targeting within the body using current techniques. These limitations with current techniques make the procedures more complicated requiring a higher level of medical and surgical skill and expertise and fully equipped operating rooms to deal with the additional risk. Despite such expensive and technically demanding expertise these procedures cause a high degree of error that lead to significant morbidity and mortality. As an example, the incidence of intracranial bleeds after any stereotactic surgery is 2% and the risk of misplacement of devices is as high as 30%.

Therefore there is a need for improved procedures to accurately determine position within the body during surgery. There is a need to allow use of high resolution images of the body interior obtained pre-procedure in conjunction with real time images available during procedures. There is a need for real-time feedback to the medical professional during the procedures. There is a need to control the introduction of air into the body during the procedures. There is a need to regulate the pressure differential across the body cavity walls during procedures. Such inventions are described here. They could be used singly or collectively to improve accuracy of placement and manipulation of medical instruments, improve the safety of the procedures and reduce surgical and other medical procedure health risks to the patient.

### SUMMARY OF THE INVENTION

We describe methods and apparatus that will enable accurate targeting and placement of a medical or surgical implement (tool) using two-dimensional or three-dimensional as required real time imaging of the body interior and thereby minimize risks to the patient. The methods include means to introduce imaging and other sensor transducers across the body wall in an accurate, clean and safe fashion. These methods significantly minimize, or eliminate, the introduction of air or other extraneous substances into the body while performing these procedures. Pressure differentials and changes across the body wall during invasive procedures are controlled and minimized. As a result there is minimal or no shift in the internal organs dimensions and positions within the body. This increases the accuracy of targeting, produces less damage to non-targeted tissue and reduces risk of infection. The apparatus and methods are related to transducers assemblies placed through the body cavity wall for real time imaging and other data acquisition, means to control the pressure differential between the body cavity and the atmosphere to limit distortion of the internal organs upon opening of the body cavity and control of contamination of the internal body cavity, imaging techniques to combine the now less distorted real-time image with pre-procedural images, and, medical or surgical tool placement effectors for accurate placement that make use of these improved real time images. Each aspect of the invention may offer improvement in medical or surgical procedures in its own right. When combined the apparatus and methods provide a singularly significant improvement in medical and surgical techniques.

### INDUSTRIAL APPLICABILITY

Examples of practical applications include:
1) Cortical and leptomeningeal biopsy: This involves taking samples of the brain or its outer coverings through a biopsy to diagnose a neurological condition using accurate stereotactic techniques.
2) Stereotactic biopsy of intracranial mass: This involves taking samples of brain tumors to diagnose, stage and treat brain tumors
3) Stereotactic placement and positioning of the stimulation electrodes for Deep Brain Stimulation This requires the accurate placement of devices and stimulators within prespecified regions of the brain.
4) Placement of depth electrodes for diagnosis of epilepsy: This involves the accurate placement of recording electrodes in specific areas within the brain to localize areas that generate seizures.
5) Stereotactic functional procedures for epilepsy, pain management, cell transplantation and gene therapy: Accurate manipulation of the and/or within the brain using stereotactic techniques to help patients with seizures, severe pain, transplantation of therapeutic cells and gene vectors in patients with neurological disorders.
6) Measurement, evaluation and decompression of raised intracranial pressure due to any cause.

In addition, the proposed methods have application in the spine for evaluation and treatment of spinal disorders that involve the spinal cord, surrounding meninges, the vertebrae, their processes, intervertebral discs and the nerve and nerve roots that exit from the spinal cord.

Though the several embodiments and descriptions presented in this document apply to targeting of a medical/surgical implement, hereinafter referred to as the “tool”, in a specified location or region of the body, hereinafter referred to as the “target”. It should be noted that the principles involved have application in any situation where a body part is enclosed within another such that imaging and access using geometrically accurate targeting would be applicable. The procedures are applicable to any of a variety of percutaneous procedures. Non-exhaustive and non-limiting examples include transcranial procedures, transthoracic procedures intrauterine procedures cardiac endovascular procedures and a variety of pulmonary and intra-abdominal procedures. Changes in pressure that deform the shape, size and position of the targeted body part can be limited and corrected using our techniques. Such applications include the beating heart that deforms with each beat and a contracting uterus during labor and delivery when stereotactic targeting of the intrauterine space becomes extremely tricky without real time imaging and distortion adjustments.

Any displacements that occur despite the use of the pressure regulating environments described here can be measured with the real time imaging and comparison across time with multiple images. Such comparison and accurate measurement of displacement will allow corrections to be applied to the stereotactic calculations and allow accurate targeting. Another situation in which these methods will advance current stereotactic techniques is when the intracranial pressure is raised (e.g. Brain tumors, intracranial bleeds, hydrocephalus, etc.). In this scenario, stereotactic procedures are fraught with the risk of brain herniation through the holes placed on the skull because the intracranial pressure exceeds atmospheric pressure. Our techniques will allow access to the brain in such a situation and allow decompression of the increased intracranial pressure in a graduated fashion such that an acute herniation of the brain can be avoided by controlling the pressure differential between the brain and atmosphere.

As a specific example, in patients with brain tumors that bleed and cause raised intracranial pressures, taking a sample of the brain tumor to decide treatment options is critical. In this scenario, stereotactic techniques that are in current use are fraught with serious danger of herniation of the brain during the procedure through the hole placed on the skull. The proposed methods will correct this issue because the hole is covered by membranes that control the pressure threshold across the skull. Thus, we can biopsy the brain tumor safely and then if necessary decompress the brain tumor and the bleed within the tumor bed such that the pressure differential can be gradually reduced. Thus the adaptive sealing device we describe here in conjunction with the real time multimodality imaging, 3D mapping of the brain and on line ability to correct trajectory will allow accurate targeting of diagnostic and therapeutic interventions in the brain and any other stereotactically accessed body part which has a differential pressure in comparison to atmospheric pressure without loss of precision of targeting and changes in pressures. The method of imaging presented here can be extended to assessment of the state of the brain.

The hollow screw assemblies and special transducer assemblies provide access points into the body. The screw assemblies and transducer assemblies are designed such that transducers are interchangeable with other transducers and with other devices. This is further enabled by the means for controlled atmosphere and pressure The transducers may monitor intracranial structure, pressure, volume, temperature, pulsitility and chemical composition of the internal structures. In addition, the hollow screw assemblies will serve as access points to the application of cortical stimulators for electrical, magnetic, pharmacological or other modalities of stimulation. These hollow screw assemblies will also allow delivery of medications, radiation and other modalities of treatment. When 2 or more hollow screw assemblies are applied to the body they could be used together in such a fashion that one is used to deliver substances into the body and another to remove substances from the inside. Using the same principle, one or more of the hollow screw assemblies could be used to deliver substances or remove substances while others are used to monitor changes inside the body. Examples of such uses include decompression of a brain tumor using chemotherapy applied directly to the tumor bed while the debris from such applications is removed via the same application hollow screw assembly or other screw assemblies. The state of the brain in terms of volume and pulsatility and also its biochemical composition then may be monitored by the other screw assemblies. Another example would be the effects of brain trauma monitored in a field hospital. It is well known that brain trauma can lead to brain swelling and the constraints of the skull causes the swelling of the brain to remain restricted in space resulting in severe damage to the brain itself. By the application of the hollow screw assemblies in the battle field, soldiers who sustain head injuries or in an emergency ambulance in the case of civilians who sustain head injuries in road traffic accidents, the status of brain swelling can be accurately monitored. Furthermore, such monitoring will determine if additional imaging is necessary so that patients can be triaged on the severity of brain trauma. The hollow screw assembly will thus serve as a window to monitor, treat or diagnose dysfunction within the cranium.

The hollow screw assembly, pressure regulating means and means to manipulate the tool remotely provide a sterile environment local to the percutaneous procedure site. Ports are provided that allow the flow of sterile air and solutions through this local controlled environment. This will in many cases enable procedures previously confined to an operating room to be done elsewhere.

In conjunction with the application of hollow screw assemblies, conventional trans-cranial ultrasound procedures via an orbital, temporal or occipital window could be used to monitor therapeutic interventions inside the cranium. For example, trans-cranial ultrasound procedures can be used to determine the location and size of the substantia nigra as has been previously described. This landmark will then be used for targeting interventions into neighboring structures like the sub-thalamic nucleus during interventional procedures (e.g. deep brain stimulation). Such trans-cranial ultrasound procedures exams could also be combined with the ultrasound information obtained through the hollow screw assembly application. Thus, such trans-cranial ultrasound procedures and percutaneous transcranial ultrasound obtained through the hollow screw assemblies could be combined and co-registered. Such co-registered data could help monitor overall brain function after the removal of the hollow screw assemblies using trans-cranial ultrasound procedures alone.

### BRIEF DESCRIPTION OF DRAWINGS

Non-limiting and non-exhaustive embodiments of the present invention are described in the following figures. Like reference numerals refer to like parts.

FIG. 1 shows multiple views of a stereotactic frame system embodiment of the invention.

FIG. 2 shows sectional views of the embodiment of FIG. 1.

FIG. 3 shows a sectional view of an embodiment that includes a transducer array.

FIG. 4 shows an embodiment that comprises a means to rotate the transducer about one axis and a multiple transducer probe.

FIG. 5 shows an embodiment that comprises a means to rotate the transducer about two axes.

FIG. 6 shows an embodiment that a comprises multiple transducers array with means to rotate about two axes.

FIG. 7 shows an embodiment that comprises external transmitters.

FIG. 8 shows an embodiment that comprises a transducer array mounted coaxially with the tool.

FIG. 9 shows a detail view of the transducer array of FIG. 8.

FIG. 10 shows an embodiment that comprises a pressure regulating means.

FIG. 11 shows an embodiment comprising an alternate pressure regulating means.

FIG. 12 shows an embodiment that comprises a pressure regulating means using a patch and dynamic seals.

FIG. 13 shows an embodiment comprising an articulated positioning tool and a transducer array.

FIG. 14 shows a sectional view of the embodiment of FIG. 13.

FIG. 15 shows an embodiment that comprises a spherical mechanism for guiding the tool.

### DETAILED DESCRIPTION

Embodiments of methods and apparatus for accurate targeting and placement of a medical or surgical instrument (tool) are described in detail. According to one embodiment a stereotactic frame is integrated with a transducer array to provide real-time feedback on tool placement. Another embodiment includes a pressure regulating means to reduce distortion in the internal organ for more accurate visualization of combined pre-operative and real time mages and more accurate tool placement. The pressure regulation means also limit contamination and enable transfer of fluids into and out of the body. Embodiments are described that include means to rotate and otherwise translate embedded transducers. Still further embodiments describe the use of externally mounted transmitters to accurately determine the location of the transducer relative to one another and to the tool. One skilled in the art will recognize that the invention can be practiced without one or more of the specific details or with other methods, components, etc. In other instances well known structures or operations are not shown or described in detail to avoid obscuring aspects of various embodiments of the invention. Reference throughout to an embodiment means that a particular feature, structure or characteristic described in connection with the embodiment is included in at least one embodiment of the present invention. Thus the phrases “in one embodiment” or “in an embodiment” in various places throughout this specification are not necessarily referring to the same embodiment. The particular features, structures or characteristics may be combined in any suitable manner in one or more embodiments.

### DEFINITIONS

The following terms are used throughout the description:

Tool—A medical device for taking some action within the body. Non-exhaustive and non-limiting examples include a drug delivery device, a suction device, a cutting or excision device either mechanical or optical, and a sensing device.
Body Wall—The exterior wall of the body, equally applicable to human or other animals. May include just the skin in percutaneous procedures or both skin and bone in for example transcranial procedures.
Body interior—The interior of the body. Non-exhaustive and non-limiting examples include the interior of the skull, the interior of the abdomen, intra-uterine spaces and the spinal cavity. The terms “body interior” and “body cavity” are used interchangeably.
Target—That location within the body interior to which the particular medical procedure is aimed. Non-limiting and non-exhaustive examples include a tumor or growth within the body, a location of particular function within the body, and a damaged site or structure within the body.
Transducer—A device for making some measurement or assessment of the body interior. Non-limiting and non-exhaustive examples include ultra-sound emitters and receivers, optical imaging devices, both optical sources and receivers.
Transducer Assembly—the transducer combined with a means to hold or mount the transducer for introduction into the body. The term transducer assembly and probe are used interchangeably.

### Transducer Assemblies

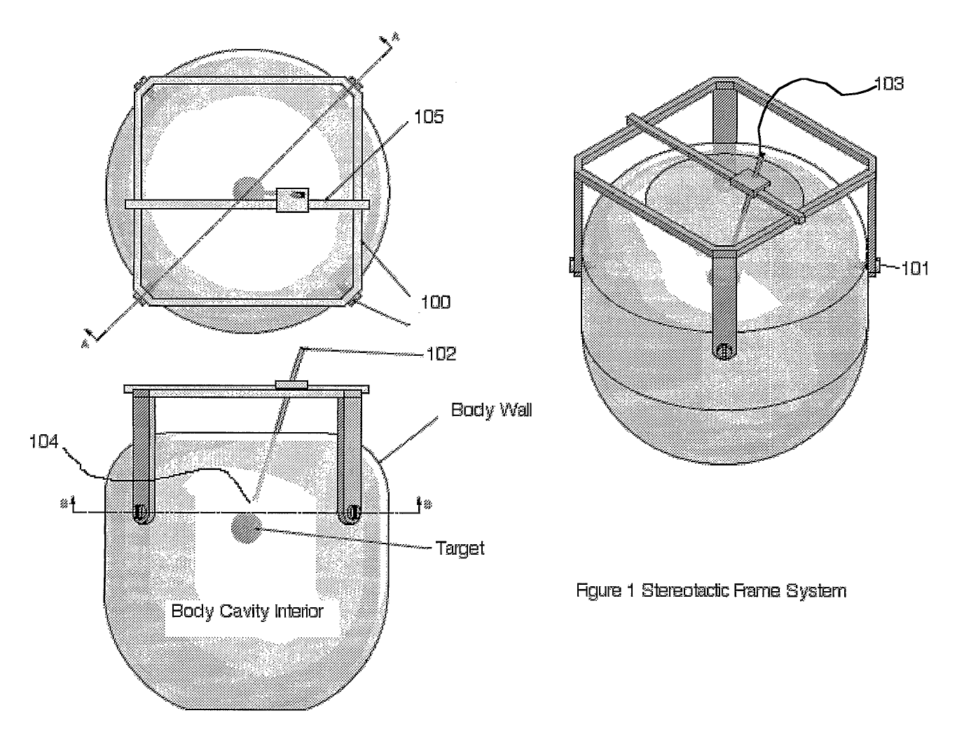

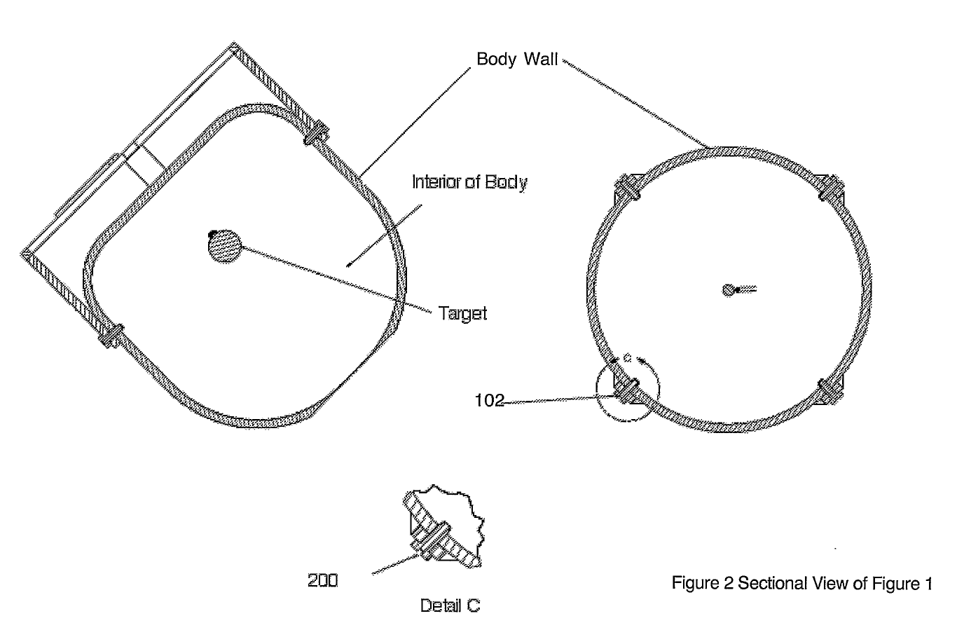

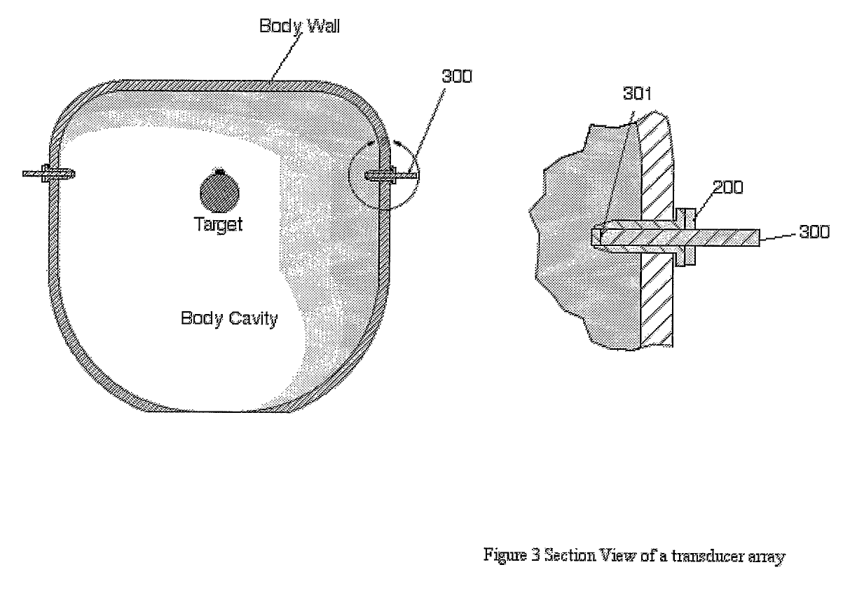

FIGS. 1 and 2 shows plan, elevation, isometric and cross-sectional views of a schematic of an embodiment used in stereotactic imaging and surgery. Holes are drilled into the body wall and a rectangular frame 100 is attached rigidly to the body wall through means of screws 101 threaded into the aforementioned holes. A magnetic resonance image, computer tomography scan, plain X-ray or other imaging of the body wall and body cavity, with the rectangular frame mounted on the body wall, is then captured and the location of the target relative to markers/fiducials on the members of the rectangular frame is then determined. The patient is then taken to the operating room and a hole drilled into the appropriate location on the body wall after which the procedure to insert the tool 102 is then initiated. The angle of approach of the tool so that the tool is guided towards the target is computed using simple trigonometry using computerized software. This mathematical information is used to reformat pre-procedural images to show in 3D the trajectory for the insertion of the tool 102. The appropriate position of the tool and angle is then set using the angular guides 103. In one embodiment the screw assemblies 101 include through-holes 200 through which are attached transducer assemblies for real-time imaging during the surgical procedure. The transducers may be emitters, receivers or both. Imaging may be performed using transducer such as ultrasound emitters and receivers, photoelectric sensors, lasers, inductive and capacitive sensors, electromagnetic radiation, x-rays, x-ray fluoroscopy, and the like. Note that there may be differences in the resolution and accuracy of the data obtained using different methods but it does not alter the basic approach presented. A transducer assembly 300 with transducer 301 is inserted through the screw assembly so that the transducer has a view of the interior of the body as shown in FIG. 3. One or more transducers may be inserted to create a transducer array for increased accuracy and resolution of the real-time image. The transducer elements 301 of the array are at the tip of the probe 300. Typically the transducer array will be located so that it is just beyond the interior surface of the body wall, or flush with it. Note that the transducer assembly 300 can either be registered using tight tolerance with respect to the through-holes 200, or the probe can be screwed in with tight tolerance if a threaded through hole is used. In this latter instance, the outer thread which is used to register the screws against the body wall can, for example, be made to run counter to the threads of the inner hole through which the ultrasonic transducer array is inserted. Thus, the external grooves in the screw will allow application of the stereotactic frame 100 on to the body part and then the inner hollow bore 200 can be exposed by removing the inner screw, such that a window into the body part is available for imaging.

By way of example for illustrative purposes the body wall in the example may refer to the skull, the body cavity would be the interior of the skull or cranium and the transducers may be ultrasonic emitters and sensors. The transducer arrays thus mounted on the reference surface are then used to identify the regions of varying densities, e.g., the skull, cartilage of the nose and other regions, the brain tissue.

In another embodiment the phase of the transducers may be varied using the known in the art, synthetic aperture method, i.e., by varying the phase of different elements of the array.

The information thus acquired on the regions of different densities is then transmitted either through a wired or wireless means of communication to a controller. Decoding software in the controller translates the information from the transducer array into a 3 dimensional array (or grid) of points representing areas of for example different densities. Hence, each point of the grid is identified by 3 dimensional coordinates and a density value. These 3 dimensional grids are then converted by modeling software in the controller to closed or open surfaces (each enclosing a volume of given density) representing bone, cartilage, tissue, etc. using appropriate surface generating approaches such as B-patches and the like. This information is used to determine the location of the target or point of interest, such as a tumor in the brain, region to be stimulated or treated, etc, relative to the reference surface, i.e., surface on which the transducers are mounted. The aforementioned transducers form the measurement system. Note that the computation of the surfaces of different densities through the use of B-patches and the like may be done in real time or offline, though real time is clearly the preferred approach. The data obtained from the transducer arrays is a discretization of the volumes and structures such as the vertebrae, ligaments, fat layers, and the like. The resolution of the grid, i.e. how coarse or fine the grid of similar density points is, can be varied by varying the sensitivity of the transducers, number of transducers, transducer arrangement, transducer geometry and scanning the transducers. The sections of the 3 dimensional grid that represent regions of the same or similar density (within a specified tolerance) are then converted by modeling software in the controller to closed or open surfaces (each enclosing a volume of the same or similar density) representing vertebrae, ligament layers, fat layers, and the like, using appropriate surface generating approaches such as B patches and the like. Note that the 3 dimensional data could also include the tool shape and volume as part of the high-density region data. Thus the data obtained is a complete 3 dimensional representation of the system. The computation of the 3 dimensional grids, and the conversion to open or closed surfaces enclosing volumes of the same or similar density are performed in the controller. The conventional stereotactic frame has 4 pin screws which when converted into hollow pin screw assemblies will provide 4 imaging windows into the body part (e.g. Interior of the skull and the brain). These 4 windows will now provide assess ports for non-invasive imaging using multiple modalities like ultrasound and fibro-optic imaging. The change in angle of the imaging probe tips that are placed into these hollow pin screws will thus allow multimodality imaging of the body part. It is not necessary to use 4 transducer arrays. It may be possible to obtain the image using 3 or 2 or 1 arrays. The number of arrays used is determined by the size of the region to be imaged, and the resolution required. Note that in many applications it may not be necessary to image the whole body cavity and body wall. It may be sufficient to image only a certain section of the body cavity for which a smaller transducer array may be required. The real-time image within the body interior may be improved with better targeting of the area of interest through the use of other transducer assemblies.

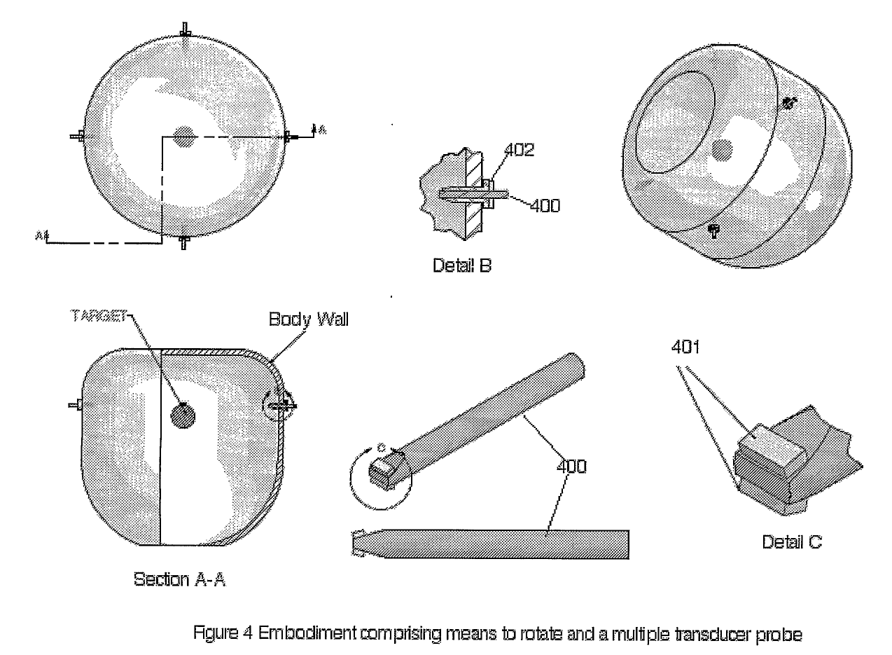

FIG. 4 shows another embodiment with a probe 400 that may be rotated about its longitudinal axis. The probe may be fitted with multiple transducers 401 to provide multimode imaging or improved imaging and targeting with a single mode. FIG. 4 shows an arrangement where two transducer arrays 401 are mounted on a probe 400 which is inserted through the hollow screw assembly 402. The probe may be rotated, either continuously or in an oscillatory manner, about the axis of the screw. The screw assembly and probe may include either a dynamic or static seal between the probe 400 and the interior wall of the screw assembly 402. By rotating, through manual or automatic means (using a motor, voice coil actuator, pneumatic actuator, hydraulic actuator, piezo electric actuator, piezo ceramic actuator and the like) the probe one can scan a larger region of the body cavity than if the transducer arrays where stationary. Oscillating and scanning probes are know to those skilled in the art to provide 3 dimensional images of the sampled region. The rotary position of the transducer can be measured using encoders (either optical, capacitive or inductive by way of example) and other feedback devices attached to or incorporated into the probe 400. Probes with two transducer arrays are used in FIG. 4 only by way of example. One or any number of transducer arrays may be accommodated. Note further that the probe may be aligned with the screw assembly using bearings or bushings or the like.

In another embodiment the rotation about the longitudinal axis is provided by fitting of coils within the screw assembly 402 and magnets within the probe 400 shaft. The screw fitted with stationary coils thereby becomes the stator, and the probe equipped with magnets, thereby becoming the rotor of an embedded electric motor that effects the rotation.

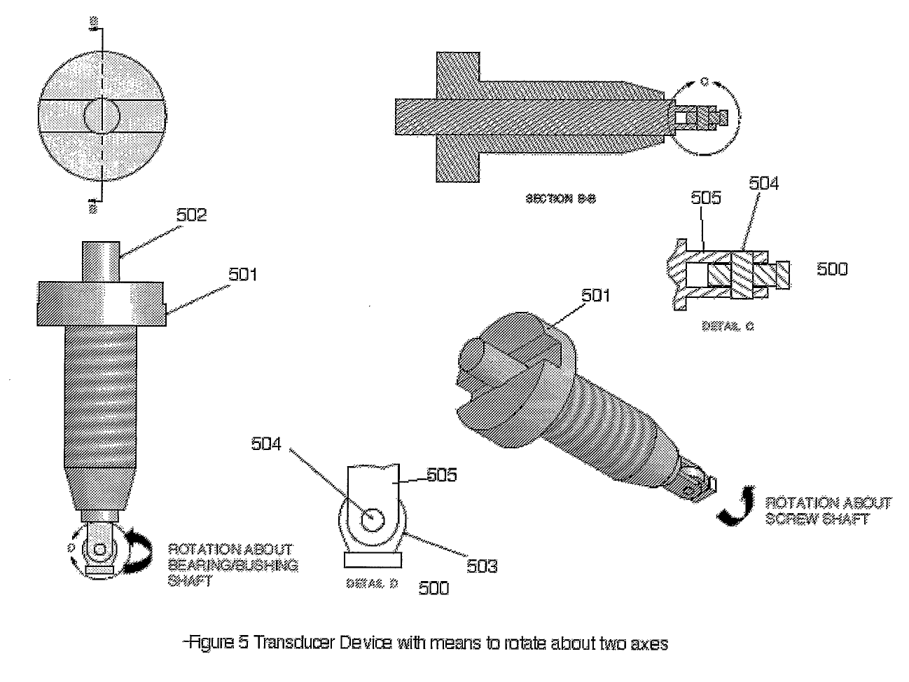

In another embodiment, shown in FIG. 5, the transducer 500 is fitted to a movable mount 503 that may be rotated not only about the shaft of the screw 502, but about a bearing/bushing shaft 504 of the transducer mount 503 perpendicular to the shaft of the screw assembly. Rotation over two degrees of freedom permits even greater ability to scan over a wide range and apply advanced scanning imaging techniques. This arrangement would, for example, allow the use of single dimensional transducer arrays instead of 2 dimensional arrays. The 2 degrees of freedom may be varied using manual means or through automatic means (such as motors, voice coil actuators, pneumatic actuators, hydraulic actuators, piezoelectric actuators, piezoceramic actuators and the like). Their positions may be measured using encoders (either optical, capacitive or inductive by way of example) and other feedback devices.

In another embodiment, the motor which drives the rotary axis that is perpendicular to the screw axis may be an integrated into the transducer mounting bracket 503. The coils of the motor may be a part of the end of the transducer mount shaft 505 that surrounds the bearing/bushing shaft 504, and the magnets may be mounted in the transducer mount shaft 503. The coils act as a stator and the magnets act as a rotor in an electric motor to effect the desired movement. The location of the coils and magnets may be interchanged as appropriate to the particular design.

In another embodiment the transducer mount may be rotated by means of a belt that transmits motion from the outside of the screw assembly to the transducer mount. Similar arrangements using other means of actuation can also be used.

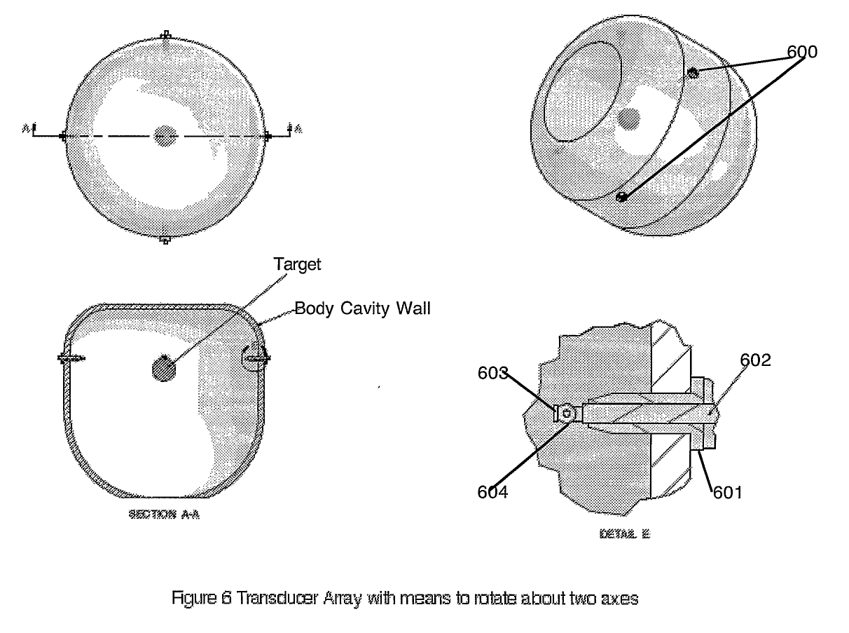

In another embodiment shown in FIG. 6, multiple transducer assemblies 600 are attached to the body wall to create a transducer array capable of high resolution imaging of the interior of the body. The assemblies may consist of the various forms of the hollow screw assemblies 601 previously discussed and probes 602. There may be single or multiple transducers 603 attached to each probe and the transducer mounts 604 may be of such a design as to allow for no rotation, single axis rotation and dual axis rotation.

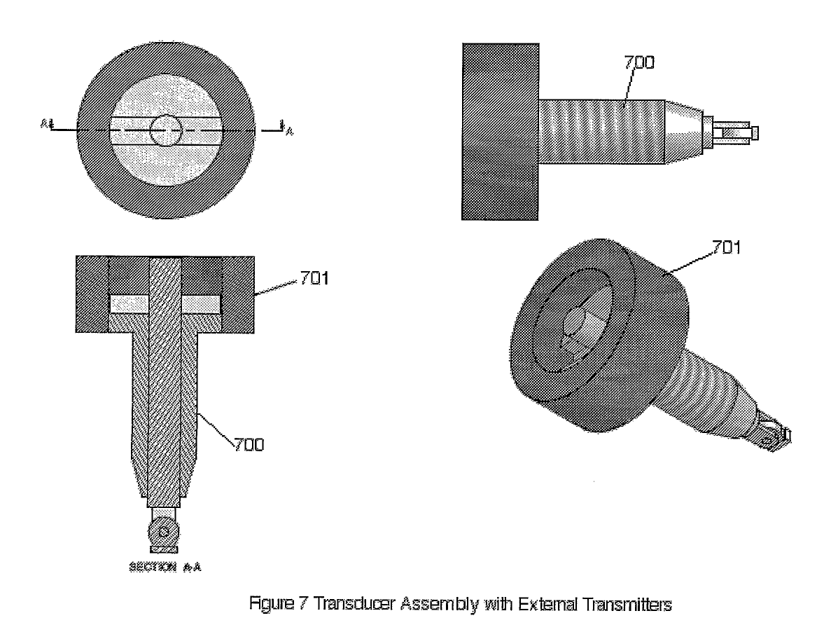

Note in the foregoing sections that the locations of the transducer assemblies are computed using pre-procedural data, i.e., locations measured before the surgical or medical procedure. However, we can measure, through triangulation, the relative positions of the transducers in real time during the procedure. Another embodiment shown in FIG. 7 shows a means to determine the relative and absolute location of each screw/probe assembly. Transmitters and/or receivers 701 which are used to compute relative position through triangulation, much like a group of satellites can use used for a global positioning system are mounted at the head of each screw 700 (note that the head of the screw remains outside the skull). These external transmitters may be electromagnetic, magnetic, inductive and the like.

In another embodiment there is an additional common transmitters mounted external to but at a known relative position to the head like a transmitter “tower” to ensure line of sight with other transmitters mounted on the head of the screws.

In another embodiment, the links 103 and base of the mechanisms 100, automated or semi automated, as well as the tool 102, are equipped with the external transmitters as well.

In another embodiment, the heads of the screws are connected to each other using mechanical linkages the joints of which are equipped with encoders. For example, two screws can be connected to each other using two links with revolute joints. Using this arrangement the relative positions of the screws and transducers can be monitored in real time by monitoring the encoder values.

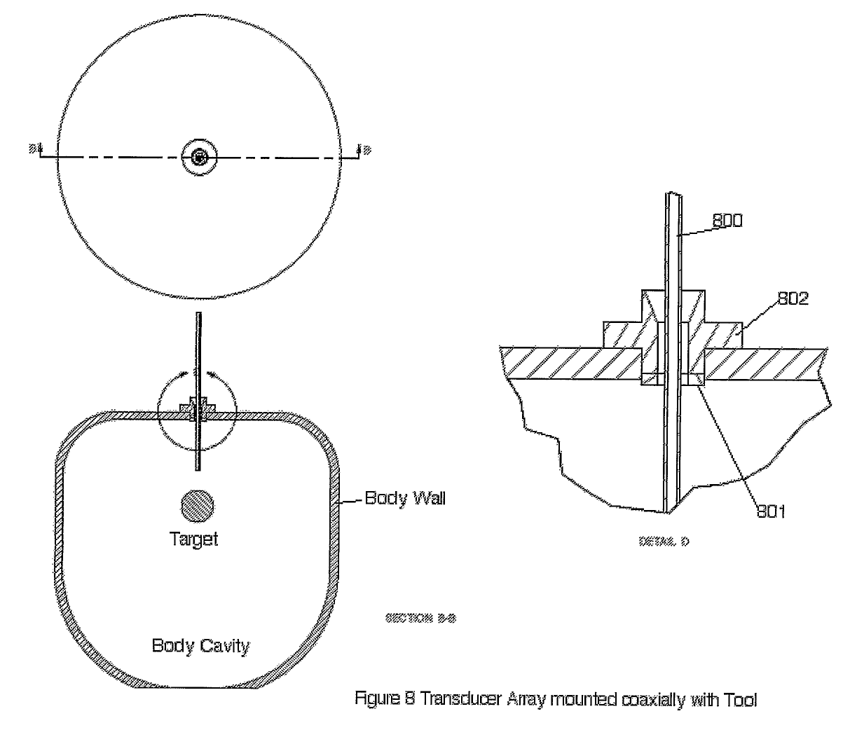

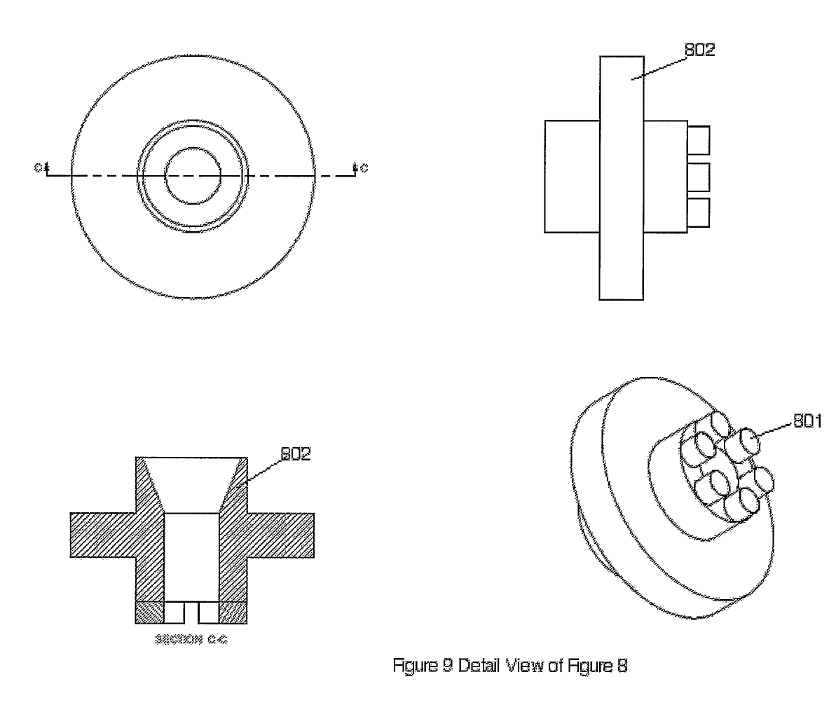

Reducing the number of incisions and intrusions into the patient is a priority for safer medical and surgical procedures. FIGS. 8 and 9 show an embodiment of the invention in which a single surgical incision and hole accommodates both the surgical tool 800 and an array of transducers 801. The multiple transducers are fit to a transducer mount 802 that also provides a seal around the operating incision. Communication to the transducers may be through connection to the top external side of the transducer mount 802 through either wired or wireless means.

### Pressure Regulating Environment

Here we describe methods and apparatus that further improve the efficacy and accuracy of the procedure by preventing the distortion of the interior organs, or body part of interest (in the case of other mentioned applications) due to pressure differentials. Note that the concept of using an enclosure to create a pressure regulated environment can be extended to the screw assemblies with through holes through which the transducer arrays are inserted. The same principle can be used to ensure that there is no deformation of the interior organs during insertion of the screw assemblies or the transducer arrays. The concept of providing pressure regulating environment can be extended to a variety of tools. For example, the inner and outer guide tubes used in deep brain stimulation. In current practice air can enter the brain in the gap/clearance between the inner and outer guide tubes. To prevent this situation the end of the outer guide tube in which the inner tube is inserted can be equipped with a valve and seals, or bellows, or the like, as described below to prevent entry of air into the brain through the guide tube. Fluids other than air can be used in the pressure regulating environment, and the choice, if available, may depend on the situation. For example, the incompressibility of saline may make pressure regulation more robust than other fluids. One possibility is to use fluids containing, for example, drugs that prevent infection.

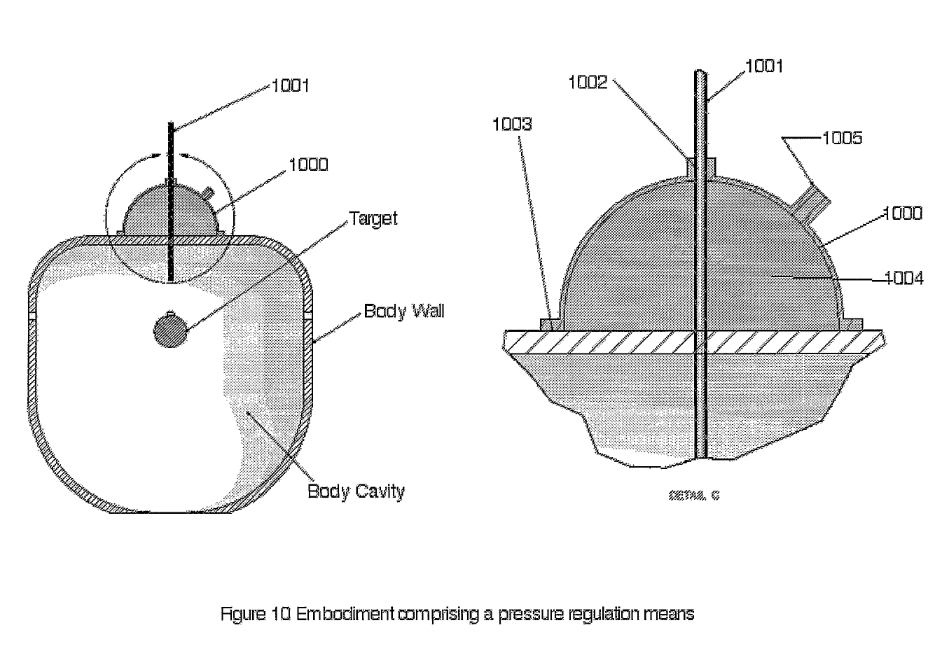

In one embodiment shown in FIG. 10 an enclosure 1000 creates a pressure regulated environment around the point of insertion of the tool 1001. The enclosure provides a seal 1002, which can be dynamic or static with the tool. It also provides a seal 1003 with surface of the body wall which can again be dynamic or static, but will typically be static (such as a compliant surface or an o-ring). The enclosure may enclose an appropriate fluid 1004, which could be air, or oxygen, or nitrogen, or saline solution, or solution with drug for disinfecting the site, or solution with drug for preventing infection, and the like. One or more pumping ports 1005 are provided for regulating the pressure of the fluid in the enclosure as required. In cases where the impact of pressure differential is not severe or critical the practitioner may choose not to use it, i.e., plug or block it off. This may also be the case when the volume in the pressure regulating environment is so small that it is not a concern. The choice of whether or not to use active control of pressure depends on how much the practitioner wishes to control the pressure differential.

In another embodiment the enclosure 1000 is compliant such as a balloon, or bellows.

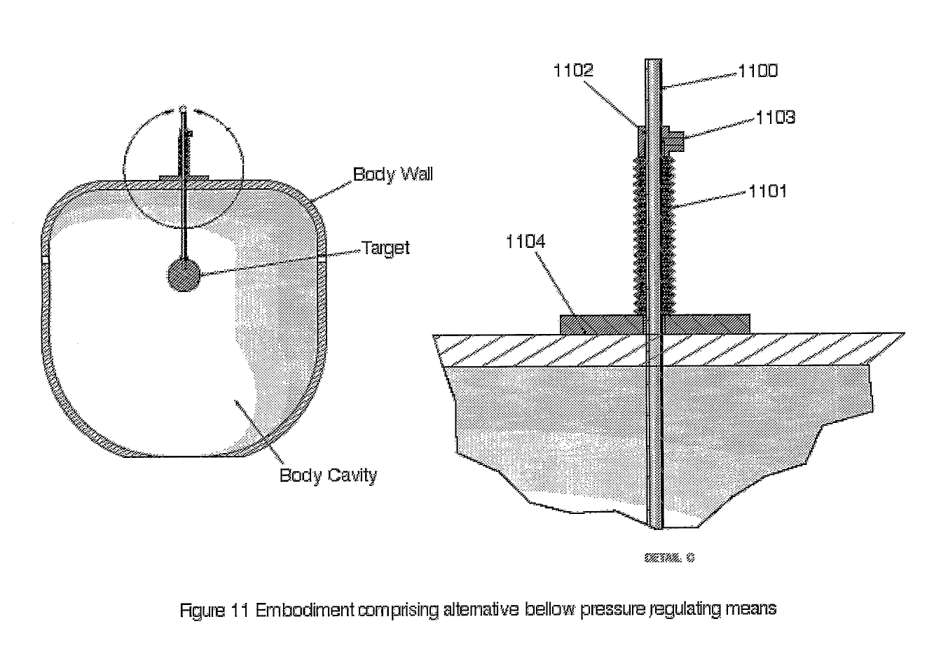

In another embodiment the pressure regulating environment is provided with a valve (typically one way), which may be either static (i.e. provided with lips, or the like, which block of air or other substances in one direction), or dynamic (actual mechanical switch). Another embodiment, shown in FIG. 11, comprises a bellows based pressure regulating device. The tool 1100 passes through a pressure fitting 1102 into the top end of the bellows 1101, which is provided with a pumping port 1103 for pressure regulation. The seal 1104 between the patient's body and the apparatus, may use gel, or glue, or some similar material to enhance the seal.

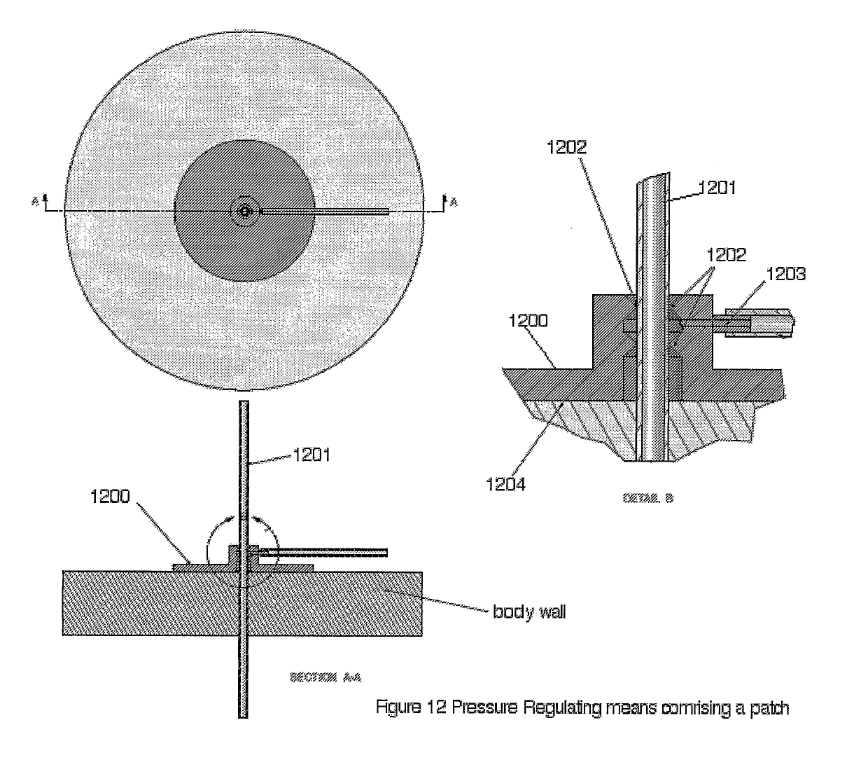

Another embodiment shown in FIG. 12 comprises a patch 1200 which adheres to the body wall forming a static seal 1204. The tool 1201 passes through the center of the patch. The embodiment further comprises one or more seals 1202. A pumping port 1203 is provided in the area between the seals 1202. This permits active pumping to control the pressure. If there are multiple lips then multiple ports can be provided if necessary, and as needed to control the pressure.

Another embodiment provides a one way valve above the lips (which could be either static or dynamic as explained before) to prevent entry of air, and the like, when the tool is inserted into the patch 1200.

### Imaging

The accuracy and sophistication of the proposed methods, as well as conventional methods, can be improved by superimposing or registering conventional images and like data obtained from conventional pre-procedural imaging techniques after an appropriate mathematical transformation as explained in the following. It is well known that the internal organs could shift and deform during surgical procedures. In conventional procedures this deformation, which corresponds mathematically to a transformation from pre-procedural data to actual data during the procedure, is not easy to predict due to lack of real time data. However with the proposed methods and apparatus, real time shapes of the body wall, shapes of the various regions of the internal organs and exact locations through registration with the transducer locations are available. Such data is also available when the pre-procedure imaging using conventional imaging techniques is performed if the screw assemblies are applied prior to the pre-procedure imaging. Thus, this data can then be used to determine the mathematical transformation from the pre-procedural data to the actual data during procedure. This transformation can then be used to map the pre-procedural data from other imaging methods to predict the real time images corresponding to those imaging methods, and that data may then be used as cross checks, for additional feedback.

In one embodiment the pre-procedural MRI data is used with real time imaging data to determine the mathematical transformation from pre-procedural data to actual data during the procedure. The resultant transformation can then be used to co-register pre-procedural images, which were obtained after placement of the screw assemblies and transducer arrays to recreate transformed pre-procedure images to be used during procedure. This permits parallel 3D display or overlap of other types of specific information that is obtained about the body part in question. Such co-registration will allow post hoc reformatting, superimposition and real time reconstruction of high resolution images with the help of the real time imaging information and thus allow further precise targeting.

### Targeting Apparatus and Procedures

The embodiments of the previous sections enable improved automated and manual placement of the surgical tool. In some cases full advantage of the invention requires improved apparatus and techniques for tool placement. In this section we describe interactive placement of the tool and diagnostic probing of the target through the use of manual and automatically controlled robotic targeting apparatus. The methods range from manual placement of the tool assisted by the real time imaging data to fully automatic placement of the tool.

The principle of the approach is to mount a mechanism that permits positioning (including orientation) of the medical or surgical tool so that it can approach and reach the target along any specified trajectory, which may either be manually computed, or automatically computed, or computed through optimization of specified criteria. The mechanism used may be any which allows positioning of the tool along the required trajectory axes.

In one embodiment shown in FIGS. 1 and 3, a stereotactic frame 100 is applied with 4 screws 101 with through holes through which ultrasonic transducer arrays 301 are inserted. The practitioner uses the real time 3 dimensional images obtained from the inserted transducers 301, as described in previous sections, to manually guide the tool to the location. The practitioner can make adjustments in the nominally computed angle of approach of the tool based on the actual real time position of the tool and target. Prior to insertion of the tool the accuracy of the nominally computed angle of the tool (hereinafter referred to as the angle of approach) is verified by superimposing the vector or array of vectors if the intended path taken by the tool is not a straight line, corresponding to the tool trajectory (which consists of positions, velocities and accelerations) on the real time images.

In another embodiment the tip 104 of tool, and possibly multiple locations on the tool 102 are equipped with transducer arrays (receivers or emitters or both) as an alternate approach to computing the exact location and angle of the tool in real time. The success of the procedure, and the wellbeing and possibly, life, of the patient is critically dependent on the successful execution of the planned trajectory of the tool. Relying on manual dexterity and control would be a risk not worth taking.

In another embodiment for safe and controlled manual placement motion of the tool is restricted so that the tool may only be manually guided along the specific trajectory, and is moreover, limit the actual deviation of tool (in terms of position, velocity, acceleration, jerk, etc) from the trajectory (including the end points along the trajectory). As noted before, the proposed methods and apparatuses use 1 or more transducers, as well as transducers of various types (such as photoelectric sensors, lasers, x-rays, etc, as noted in previous sections) at appropriate locations on the body wall. This method, therefore, permits elimination of the cumbersome stereotactic frame currently used, and permits real time 3D imaging of the body interior. The real time images permit real time automatic adjustment of the tool location and trajectory and can potentially dispense with the need to restrain the patient to the procedure table, as is currently required by current stereotactic frames or frameless techniques but require restraining the patient to the procedure table. Further the ability to make corrections to tool locations and in real time is extremely critical in patients with tremor disorders that make the body part oscillate in space and time. Such motion will not hamper stereotactic procedures using the proposed methods and apparatuses due to the ability to monitor in real time and move, correct and compensate the tool trajectory (as described in previous sections) in real time. The use of pressure regulating environments further improves performance.

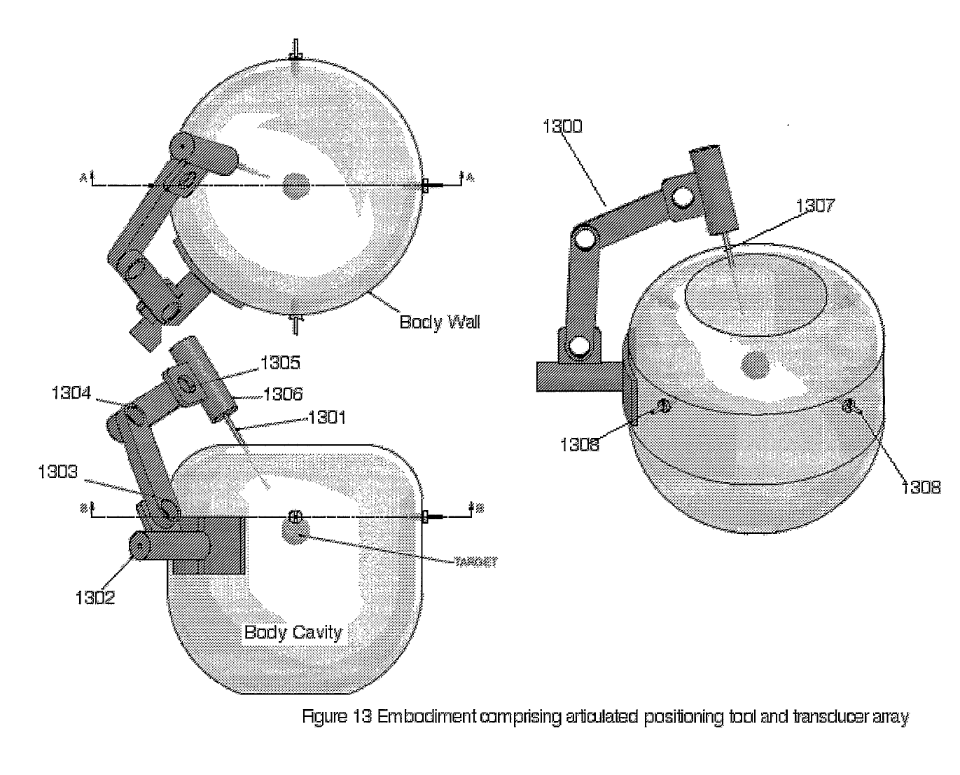

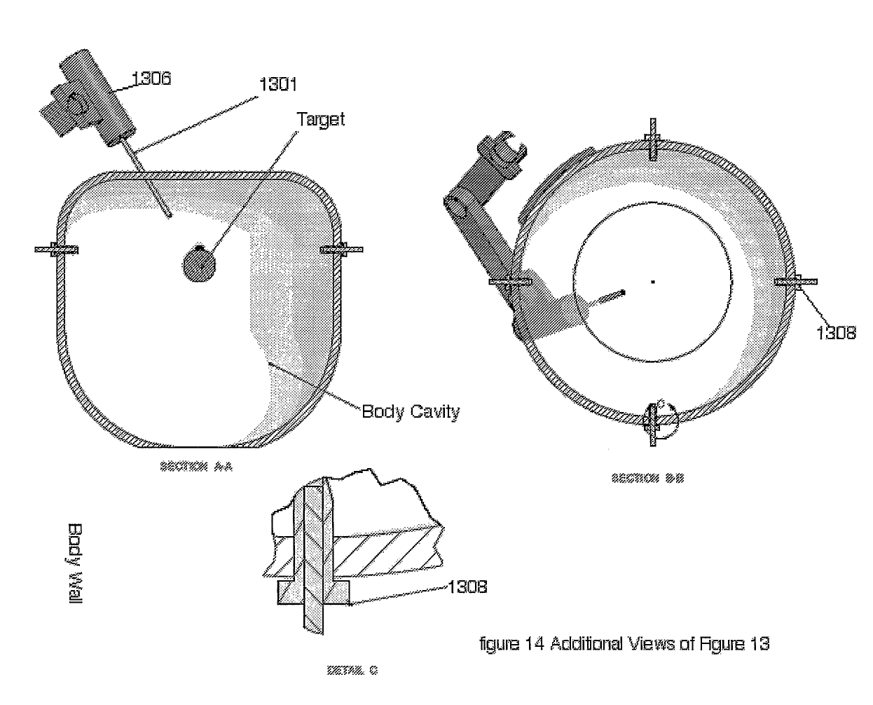

In one embodiment, shown in FIGS. 13 and 14, an articulated mechanism 1300, is used to move the tool 1301 to the target site within the body. The mechanism shown has 4 rotary axes 1302-1305 for positioning and orienting the end effector 1307 at the appropriate location. Another axis 1306 which could be linear or rotary with transmission, such as a rack and pinion, belt drive, ball screw, voice coil actuator, solenoid, etc, moves the tool in the direction of the end effector 1307. This mechanism is equipped with motors and encoders at the joints. Position feedback of the end effector and tool tip is through means of the images provided by the implanted transducers 1308.

In another embodiment each axis mechanism 1302-1306 is further provided with brakes, analog and digital IO (input-outputs) for limits switches, and feedback of other types, as in typical force control applications. The purpose of the motors would be to provide force control so that there is no net force, or minimal net force, on the tool and end effector, permitting easy maneuvering of the tool. The encoders would be used to provide information of the deviation of the tool from the specified trajectory and when the actual trajectory deviates from the specified trajectory the tool would be braked, or held in position, and, for example, an alarm sounded.

In another fully automated embodiment, the movement of the tool would be accomplished using motors, such as servo motors, mounted on the axes of the linkage. The practitioner would be able to manually intervene if necessary. The joints may be driven using servo rotary motors, linear motors, pneumatic actuators, hydraulic actuators, piezoceramic motors and actuators, electromagnetic actuators such as rotary and linear solenoids, etc.

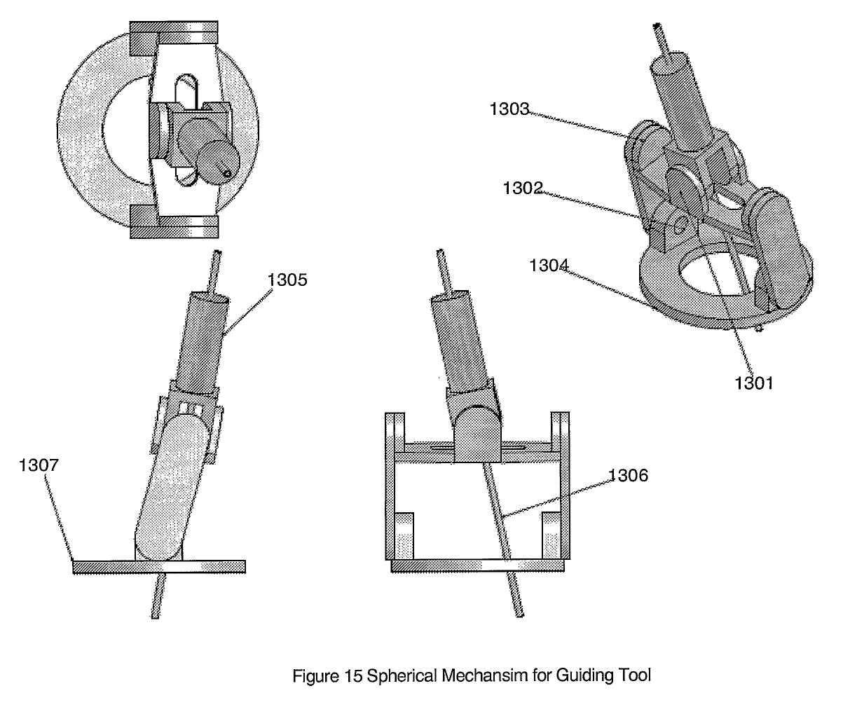

Another embodiment, shown in FIG. 15, is comprised of a compact spherical mechanism of 3 rotary axes, 1501-1503 used to fix the insertion location on the skull and the angle of the tool 1506, and 1 linear axis 1505, for advancement of the tool.

In another embodiment, a membrane is adhered to the exterior body wall, or a section of it. The membrane might have any appropriate two dimensional pattern which could be etched or imprinted and behave like a two dimensional encoder or bar code reader. The base mechanism 1507 is a moving member which tracks the contours of the body wall, either using a track mechanism, or the like which provides ability to move along two dimensions. The position of the base is determined relative to the patterned membrane with its 2 dimensional pattern.

In another embodiment the practitioner would use semiautomated or automated means to move the tool from a remote location. Communication is effected by means of the internet, wireless network, and otherwise. Note that with high bandwidth this may be done in real time. If bandwidth proves be a limitation then it may be required that the practitioner first plan a move, review it in simulation mode, and then execute it with a separate command.

Approaches without Imaging

The transducer design, the pressure regulation and the targeting apparatus and procedures provide improved means for providing and using real-time imaging during surgical procedures. However advantages can be obtained using the inventions without real-time imaging. The procedures generally consist of the following steps:

1) Estimate the dimensions of the body contours (external, or both external and internal) relative to the base any of the surgical mechanism described above and shown in FIGS. 1, 13, 14 and 15 (referred hereinafter as the mechanism coordinates), using the kinematic transformation from the base of the mechanism to the tool tip.
2) Transform the pre-procedural image data obtained, which includes target location, to the mechanism coordinates.
3) Use any of the pressure regulating enclosures described above and shown in FIGS. 10-12 to ensure no, or minimal, distortion
4) Move the tool to the target using the surgical mechanism

In one embodiment, the targeting mechanism determines the coordinates of different locations on the exterior body wall by touching the tool tip at various locations of the body wall, much like a coordinate measuring machine is used to inspect the dimension of machined parts. Note that the term “touching” here also includes driving the tool tip beyond the skin till it hits the outer surface of the bone such as the skull. To facilitate this measurement step the tool tip may be equipped with a sensor such as a proximity sensor, contact sensor, and the like. The rest of the steps are as outlined in steps 2-4 immediately above.

In another embodiment the sensors, such as photoelectric sensors, laser sensors, magnetic sensors, inductive sensors, electromagnetic sensors and the like are mounted at various locations on the skull, as well as on the base of the mechanism and possibly at other locations on the mechanism. The locations of these sensors relative to each other are obtained from the pre-procedural data. Using triangulation, the location of the different links of the mechanism as well as the end effector relative to this coordinate system are known. The rest of the steps are as outlined in steps 2-4 immediately above.

Another embodiment is a blind till insertion option. In this instance transducers are mounted at one or more locations on the tool. Here the estimation of the body wall contours are done as before. Then once the tool is inserted real time images are obtained from the point of view of the tool coordinate system. This allows obtaining real time images after insertion and eliminates the need to drill holes to insert the arrays and at the same time provides real time images during procedure.

### CONCLUSIONS

Apparatus and methods are described providing improved surgical and similarly invasive procedures. The components may be used individually or collectively as a system. They consist of new apparatus for insertion of transducers and transducer arrays into the body cavity, apparatus and procedures for controlling pressure differential between the body cavity and the atmosphere, imaging techniques to combine the now available real-time images with pre-procedure imaging, and robotic placement techniques that when combined with all of the above provide improved surgical and medical procedures.

What is claimed is:

1. A device for placement and operation of transducer devices used in the measurement and observation of the interior of a body cavity comprising a screw device having a head and distal end and a center longitudinal cavity and a transducer probe having a longitudinal axis replaceably passing through said central longitudinal cavity, said transducer probe fitting sufficiently snugly within said center longitudinal cavity to limit leakage in to and out of the body cavity and at least one transducer attached to the probe to extend to said distal end of said screw device and said head of said screw device remaining outside of the body during use.

2. The device of claim 1 further comprising a rotating means about the said longitudinal axis of said transducer probe.

3. The device of claim 1 further comprising a means for movement of the transducer on at least one axis perpendicular to the longitudinal axis of the transducer probe.

4. The device of claim 2 said rotating means further comprising a coil incorporated into the said screw device and a stator incorporated in the transducer probe said rotor and said stator acting in concert as a electric motor to effect said rotation.

5. The device of claim 3 said movement means further comprising a stator incorporated in a mounting shaft for said transducer and a rotor incorporated in a mounting bracket for said shaft, said rotor and said stator acting in concert as an electric motor to effect said movement.

6. The device of claim 1 where the transducer device is an imaging device.

7. The device of claim 1 further comprising a locator means attached to said head of said screw device.

8. A device for placement and operation of a medical tool and transducer devices for medical or surgical procedures comprising a sealing assembly having an external surface and an internal surface and a sealable port centrally located and connecting said external and internal surfaces through which may be passed a medical tool and a means to attach transducers to said interior surface.

9. A device for protection of the interior of a body cavity subject to a surgical or other medical procedure from the atmosphere or surrounding environment comprising a chamber through which a surgical tool or other probe may pass and remain externally operational, a seal between said chamber and the external body surface surrounding the body cavity and at least one seal between said surgical tool or other probe and said chamber.

10. The device of claim 9 where the chamber further comprises means to change dimensions including internal volume of said chamber.

11. The device of claim 9 further comprising a port means and a pumping means attached to said port means for transfer of fluids into and out of said chamber.

12. A device for protection of the interior of a body cavity subject to a surgical or other medical procedure comprising a patch having an upper surface and a lower surface, said lower surface statically sealed to the exterior body cavity wall, a sealable port passing through said upper and lower surfaces through which a surgical tool or other probe may pass and remain externally operational, and a seal between said tool and said port.

13. The device of claim 12 further comprising at least two seals between said tool and said upper surface and a pumping means attached to said upper surface located between said at least two seals.

14. The device of claim 12 where the patch is composed of flexible material.

15. A system for performing a medical or surgical procedure on a target within a body cavity comprising at least one transducer probe assembly with means to replaceably introduce a transducer into the body cavity through the wall of the body cavity, a sealing means at the body cavity wall to introduce a medical tool into the body cavity, a means to manipulate and control said tool externally to the body cavity to effect the procedure at the target, a stereotactic means to guide said tool to the target, wherein said guiding means comprises coordinates of the target obtained from pre-procedure images corrected by transformations to coordinates obtained from images of the target obtained during the procedure, said images during the procedure obtained from said at least one transducer.

16. The system of claim 15 wherein said transducer probe assembly further comprises means to rotate said transducer about at least one axis.

17. The system of claim 15 where said sealing means further comprises a means to regulate the pressure differential across the body wall.

18. The system of claim 15 where said sealing means further comprises a means to pump fluid in to or out of said sealing means.

19. The system of claim 15 wherein said means to manipulate and control the tool is an articulated arm.

20. The system of claim 15 wherein said means to manipulate and control the tool is a spherical mechanism comprised of three rotary axes and one linear axis to which the tool is attached.

21. The system of claim 19 wherein said articulated arm is motorized.

22. The system of claim 21 where said motorized articulated arm is automatically controlled by a computer system which advances the tool to the said corrected target coordinates.

23. The system of claim 21 where said motorized arm is manually guided to said corrected target coordinates.

24. The system of claim 23 further comprising means to restrict the articulated arm to a path to said corrected target coordinates.

25. A system for performing a medical or surgical procedure on a target within a body cavity comprising a sealing means at the body cavity wall to introduce a medical tool into the body cavity, a means to manipulate and control said tool externally to the body cavity to effect the procedure at the target, a stereotactic means to guide said tool to the target, wherein said guiding means comprises coordinates of the target obtained from pre-procedure images.

26. The system of claim 25 wherein said sealing means further compromises a means to regulate the pressure differential across the body wall.

27. The system of claim 25 wherein said sealing means further compromises a means to pump fluid in to or out of said sealing means.
